(19) United States
(12) Patent Application Publication
(10) Pub. No.: US 2007/0191816 A1
(43) Pub. Date: Aug. 16, 2007

(54) Radio frequency induced drug elution

(75) Inventors: Niall Behan, Kilcolgan (IE); Anthony Malone, Miltown Malbay (IE); Aiden Flanagan, Kilcogan (IE); Barry O'Brien, Cappagh Road (IE); John Motherwell, Kinvara (IE); David McMorrow, Fort Lorenzo (IE)

Correspondence Address: KENYON & KENYON LLP, ONE BROADWAY, NEW YORK, NY, 10004, US

(73) Assignee: Boston Scientific Scimed, Inc., Maple Grove, MN (US)

(21) Appl. No.: 11/353,800

(22) Filed: Feb. 13, 2006

(51) Int. Cl. A61K 9/22

(52) U.S. Cl. 604/890.1

(57) ABSTRACT

A medical device including at least one reservoir, adapted to contain a material, such as a therapeutic agent, and sealed by a cover. The reservoir is openable, so as to release the material, for example, by (i) disintegration of the cover, (ii) movement of the cover to an open position, and/or (iii) a change of composition or properties of the cover allowing the material to pass therethrough. The power required to open the reservoir is provied by a current induced in a coil associated with the medical device by a change in magnetic flux in the area of the medical device.

### FIELD OF THE INVENTION

The present invention relates to an apparatus and method for releasing one or more therapeutic agents from a medical device.

### BACKGROUND OF THE INVENTION

U.S. Pat. Nos. 5,797,898, 6,123,861, 6,491,666 and 6,537,256, to Santini Jr. et al., herein incorporated by reference in their entirety, disclose a microchip drug delivery device that provides active timed release of a drug. The microchips control both the rate and time of release of multiple chemical substances and allow for the release of a wide variety of molecules in either a continuous or pulsatile manner. A material that is impermeable to the drugs or other molecules to be delivered and the surrounding fluids is used as the substrate. Reservoirs are etched into the substrate using either chemical (wet) etching or ion beam (dry) etching techniques well known in the field of microfabrication. The molecules to be delivered are inserted into the reservoirs by injection or spin coating methods in their pure form or in a release system. The physical properties of the release system control the rate of release of the molecules. The reservoirs can contain multiple drugs or other molecules in variable dosages. The filled reservoirs can be capped with materials that either degrade or allow the molecules to diffuse passively out of the reservoir over time or materials that oxidize and dissolve upon application of an electric potential. Release from an active device can be controlled by a preprogrammed microprocessor, remote control, or by biosensors.

Microchip devices have numerous in vitro and in vivo applications. The microchip can be used in vitro to deliver small, controlled amounts of chemical reagents or other molecules to solutions or reaction mixtures at precisely controlled times and rates. Analytical chemistry and medical diagnostics are examples of fields where the microchip delivery device can be used. The microchip can be used in vivo as a drug delivery device. The microchip can be implanted into a patient, either by surgical techniques or by injection. The microchip provides delivery of drugs to animals or persons who are unable to remember or be ambulatory enough to take medication. The microchip further provides delivery of many different drugs at varying rates and at varying times of delivery.

The microchip may also be incorporated into a medical implant, such as catheters, guide wires, balloons, filters (e.g., vena cava filters), stents, stent grafts, vascular grafts, intraluminal paving systems, implants and other devices. Such medical devices are implanted or otherwise utilized in body lumina and organs such as the coronary vasculature, esophagus, trachea, colon, biliary tract, urinary tract, prostate, brain, and the like. Medical implants are used for a number of medical purposes, including the reinforcement of recently re-enlarged lumens, the replacement of ruptured vessels, and the treatment of disease such as vascular disease by local pharmacotherapy, i.e., delivering therapeutic drug doses to target tissues while minimizing systemic side effects.

There is a need, however, for a power source capable of powering the above-described microchips without interfering with functioning of the device. Santini Jr. et al. disclose a power source connected to the microchip device itself. However, location of the power source on the microchip device necessarily increases the size of the microchip and requires that the power source be coated or otherwise protected from the environment which the microchip is exposed. Further, location of the power source on a device, such as a medical implant, incorporating the microchip may interfere with the functioning of the medical implant, etc.

### SUMMARY OF THE INVENTION

The present invention relates to a device including at least one reservoir, fillable with a material, such as a therapeutic agent, and sealed by a cover. The reservoir is openable, so as to release the material, for example, by (i) disintegration of the cover, (ii) movement of the cover to an open position, and/or (iii) a change of composition or properties of the cover allowing the material to pass therethrough. The power required to open the reservoir is provided, consistent with Faraday's law, by a current induced in a coil associated with the device by a change in magnetic flux in the area of the device.

An exemplary embodiment of the present invention includes at least one reservoir fillable with a material, such as a therapeutic agent, a cover configured to seal the at least one reservoir in a first state and to allow the material to exit the at least one reservoir in a second state, and at least one coil. The cover is configured such that it transitions between the first and second states when the coil is exposed to a magnetic flux. The magnetic flux may have a radio frequency and may be time varying.

The cover in the second state may (i) be at least partially disintegrated, (ii) completely cover the reservoir but allow the material to pass directly through it, (iii) be shifted away from or towards the reservoir so as to unseal the reservoir, and/or (iv) be fully intact but have at least one smaller dimension than in the first state.

The coil may be connected across the cover such that a current induced in the coil by the magnetic flux passes through the cover. The current may be rectified to produce a DC current through the cover.

The device may include a substrate in which the at least one reservoir is formed.

The device may be a stent. The stent may have a coil configuration and at least a portion of the stent itself may form the coil that is used to transition the cover between the first and second states. Alternatively, the coil that is used to transition between the first and second states may be attached to a surface or a body of the stent, or wrapped around the stent.

The device may include another coil. The at least one coil and the other coil may be configured such that they each conduct a different level of current upon exposure to the same magnetic flux.

The device may include a second reservoir covered by a second cover configured to seal the second reservoir in a first state and to allow the material to exit the second reservoir in a second state. The first cover may be configured to transition between its first and second states when a first current passes through it, and the second cover may be configured to transition between its first and second states when a second current different from the first current passes through it. The second reservoir may be filled with a therapeutic agent different from the therapeutic agent in the first reservoir.

The device may include at least one switch configured to control electrical communication between the reservoir and the coil.

The device may include a controller configured to control the at least one switch, for example, made from a semi-conducting material.

The controller may have an input from another coil used to instruct the controller as to when to open and close the at least one switch.

The device may include a sleeve, for example, a polymeric sleeve, in which the least one reservoir is formed.

In an exemplary method of the present invention, the above described device may be used to control the release of a material from the device by exposing it to a magnetic flux, for example, a time varying magnetic flux.

### BRIEF DESCRIPTION OF THE DRAWING

The present invention will become more fully understood from the detailed description given hereinbelow and the accompanying drawing, which is given by way of illustration only and wherein:

FIG. 1A is a perspective view of an exemplary embodiment of a stent according to the present invention.

FIG. 1B is a perspective view of an exemplary embodiment of a stent according to the present invention.

FIG. 2 is a transverse cross section of the stent of FIG. 1A taken along lines 2-2 in FIG. 1.

FIG. 3 is a longitudinal cross section of the stent of FIG. 1A taken along lines 3-3 in FIG. 1.

FIG. 4A is an exemplary embodiment of the substrate in FIG. 1A including a heater.

FIG. 4B shows the substrate of FIG. 4A with the cover in an opened state.

FIG. 5A is an exemplary embodiment of the substrate in FIG. 1A with a cover, shown in a closed state, made from a piezoelectric material.

FIG. 5B shows the substrate of FIG. 5A with the cover in an opened state.

FIG. 6A is an exemplary embodiment of the substrate in FIG. 1A with a cover, shown in a closed state, pivotally controlled by a motor.

FIG. 6B shows the substrate of FIG. 6A with the cover in an opened state.

FIG. 7A is an exemplary embodiment of the substrate in FIG. 1A with a cover locked in a closed state by a magnet.

FIG. 7B shows the substrate of FIG. 7A with the cover in an opened state.

FIG. 8 is a transverse cross section of an exemplary embodiment of a stent in accordance with the present invention with a reservoir embedded in the stent.

FIG. 9 is an exemplary embodiment of a stent in accordance with the present invention with a coil wrapped around the surface of the stent.

FIG. 10 is an exemplary embodiment of a stent graft of the present invention.

FIG. 11 is an exemplary embodiment of a reservoir and cover of the present invention controlled by a switch and microprocessor.

FIG. 12 is an exemplary embodiment of the present invention including multiple reservoirs and covers, each individually controlled by a separate switch.

### DETAILED DESCRIPTION OF THE INVENTION

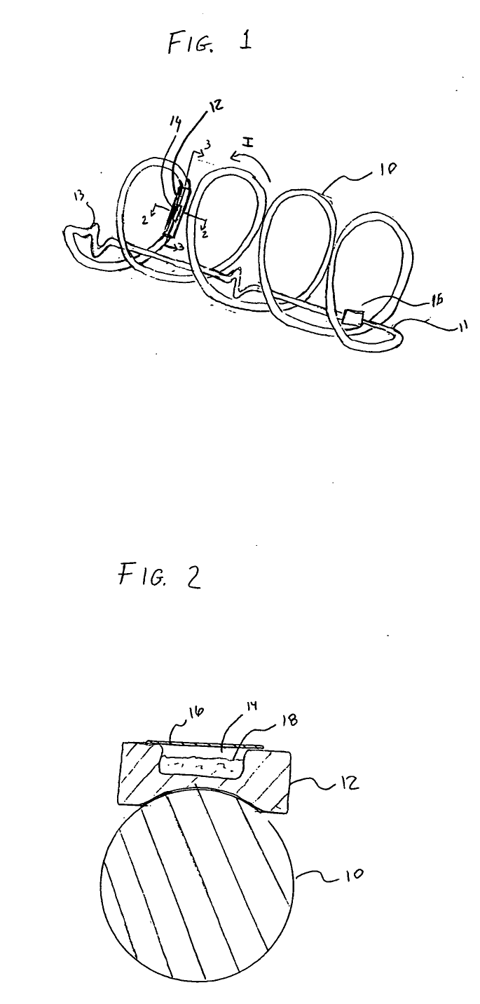

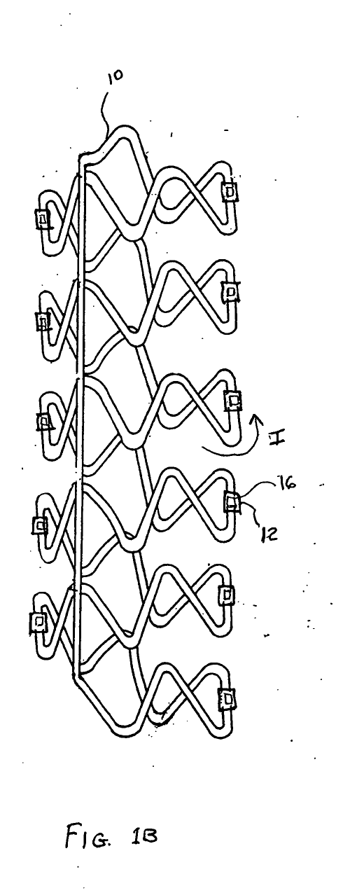

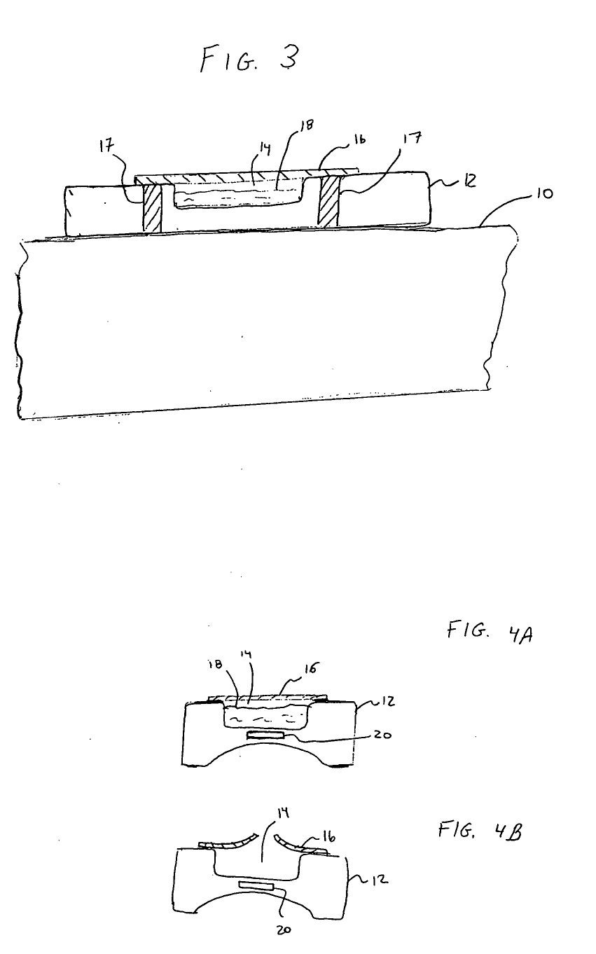

FIG. 1A illustrates an exemplary embodiment of a stent 10 according to the present invention. A substrate 12 having a reservoir 14 therein is connected to the stent 10. As can be seen in the transverse and longitudinal cross sections of the substrate shown FIGS. 2 and 3, a cover 16 secured over the reservoir 14 is used to seal material 18, such as a therapeutic agent or drug, in the reservoir 14. Cover 16 is in electrical communication with the stent 10 via electrical contacts 17. Stent 10 itself is in the shape of a coil and forms a closed loop. Portion 11 of stent 10 may have a deformable sinusoid or zig-zag portions 13 allowing the stent 10 to change dimension. Upon exposure of the stent 10 to a predetermined magnetic flux, a current, I, develops in the body of the stent 10 and passes through the cover 16 via electrical contacts 17. The current disintegrates cover 16 releasing the material 18 from confinement in the reservoir 14.

The properties of the stent 10, e.g., number of coils, size of coils, conductivity, etc., may be specifically chosen so as to assure a predetermined level of current across the cover 16 upon exposure to a predetermined magnetic flux. For example, the stent 10 may be designed to avoid release of material 18 upon exposure to magnetic flux generally associated with noise, e.g., approximately 50 Hz. Stent 10 may also include a capacitor 15 connected across its ends, which may be used to control the resonant frequency of the stent 10. For example, the stent 10 may be tuned to 64 MHz emitted by an MRI machine and, thus, when exposed to electromagnetic radiation of this frequency will cause current to run across cover 16 and release the material 18. Conversely, the stent 10 may be designed with a resonant frequency unlike the electromagnetic frequencies emitted by an MRI machine (64 and 128 MHz) so as to avoid accidental release of material 18 by an MRI machine during a diagnostic scan.

FIG. 1B illustrates another embodiment of a stent in accordance with the present invention with a plurality of covered reservoirs 14. As with the stent of FIG. 1A, a body of the stent forms a closed loop. The stent illustrated in FIG. 1B has an underlying structure that is similar to a stent illustrated in U.S. Pa. No. 5,653,727. Although not shown, the stent illustrated in FIG. 1B, similar to the stent of FIG. 1A, may also include a capacitor connector across its ends to control the resonant frequency of the stent 10. Persons of ordinary skill in the art will recognize that other stent structures and geometries may be used.

In an exemplary method of operation, a patient having the stent 10 implanted in his or her body is exposed to a magnetic flux generated, for example, by an MRI machine. The magnetic flux may be generated, for example, by the electromagnetic radiation of radio frequency created by the MRI machine. The magnetic flux generates a current in the stent 10 thus triggering the release of the material 18, e.g., the therapeutic agent, in the reservoir 14. The therapeutic agent may be released, for example, to a vessel wall or into the blood stream of the patient. The magnetic flux may, for example, be time varying and at any frequency capable of generating a predetermined current in the stent 10.

Cover 16 may be made from any material capable of sealing the material 18 in reservoir 14 and capable of disintegrating upon exposure to a current developed in the stent 10 when the stent 10 is exposed to a predetermined magnetic flux. For example, the cover 16 may be made from a metal, such as copper, gold, silver, magnesium and/or zinc, or from a polymer. The cover 16 may also be made from an electro-activated polymer configured to change properties upon application of a current (rather than disintegrating) so as to allow the material 18 in reservoir 14 to pass directly through the cover 16. Examples of electro-activated polymers include polypyrole and Perfluorinated Ion exchange membrane metal composites (IPMC) using Nafion® film from DuPont.

In an exemplary embodiment, the substrate 12 or stent 10 may include a means for changing the temperature of material 18, such as a resistive heater or a cooler. For example, upon application of heat to the material 18, the material 18 may expand and, thereby, rupture cover 16 thus releasing material 18 from reservoir 14. FIG. 4A is a transverse cross sectional view of the substrate 12 including a heater 20 connected to the substrate 12 and configured to heat and cause the expansion of material 18 for purposes of rupturing cover 16. FIG. 4B illustrates the state after rupture of cover 16.

In an exemplary embodiment, the material 18 may be heated by heater 20 so as to cause diffusion of the material through cover 16 out of reservoir 14 without rupture of cover 16.

In an exemplary embodiment, the substrate 12 may be made from a piezoelectric material. Upon application of a current across the substrate 12, the substrate 12 may expand, for example, along the longitudinal axis of a stent strut, thereby pulling or tearing open the cover 16, which does not expand as a result of the generated current. The cover 16 maintains contact with the substrate 12 along the edges of the cover 16.

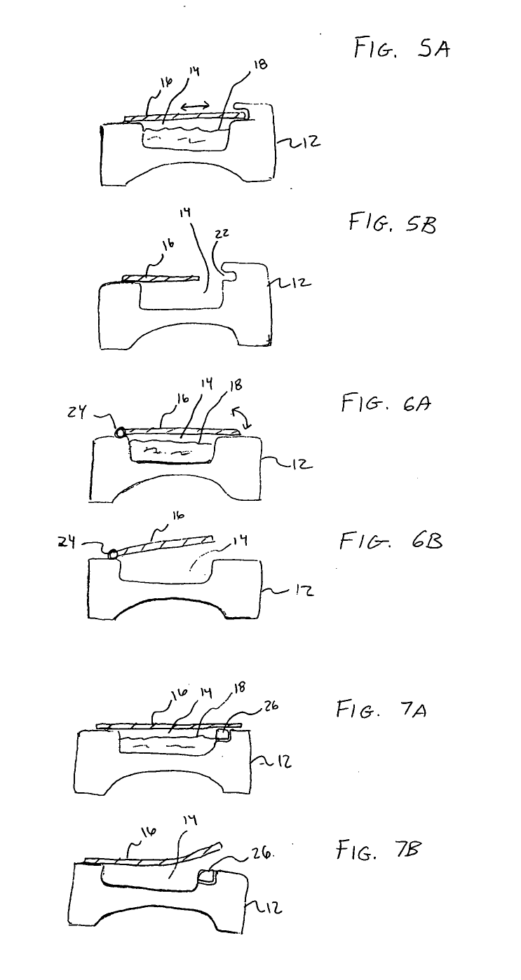

In an exemplary embodiment, cover 16 itself may be made from a piezoelectric material. As shown in FIG. 5A, the cover 16 may be connected to the substrate 12 on one end and slidingly received in a slot 22 on an opposite end. Upon application of a current across cover 16, for example, on opposite ends of the left side of the cover 16, the cover may shrink in width and move to an open position, shown in FIG. 5B.

In an exemplary embodiment shown in FIG. 6A, the cover 16 may be pivotally connected to the substrate 12 and controlled by a miniaturized motor 24. The cover 16 may pivot up away from the substrate 12, pivot down into the reservoir 14 or may move along the plane of the cover 16 so as to expose the material 18 in the reservoir 14. FIG. 6B shows the cover 16 in an opened position. Cover 16 may be configured so as to be sufficiently rigid so as not to bend or flex towards the reservoir 14 when in the opened position.

In an exemplary embodiment shown in FIG. 7A, the cover 16 may be pivotally connected to the substrate 12 and locked to the substrate 12 via a magnet 26 connected to the substrate 12. The cover 16 may also be maintained in the closed position using a spring (not shown). The cover 16 may be made from a material, such as a magnetized metal, that is (i) attracted to magnet 26, thereby locking the cover 16 in place over the reservoir 14, and (ii) repelled into an open position by a magnetic field created when current runs through stent 10, thereby releasing the material 18 in reservoir 14. The magnetic field used to open cover 16 may be sufficiently strong to flex cover 16 into the open position shown in FIG. 7B.

In an exemplary embodiment, the reservoir 14 may be embedded directly in a stent as opposed to in a substrate connected to the stent. FIG. 8 shows a transverse cross section of a stent 10 incorporating such an embedded reservoir 14. As with the embodiment of FIG. 1, cover 16 seals reservoir 14, and exposure of the stent 10 to a magnetic flux generates a current, I, which opens the reservoir 14 via disintegration of cover 16 or via any of the methods discussed above in regard to FIGS. 4A-7B. In an exemplary embodiment the diameter or width of the stent wire or struts is 150 microns, and the reservoir 15 has a depth of up to 100 microns.

In an exemplary embodiment, rather than using or in addition to using the stent body as a current generating coil, one or more coils may be connected to or adhered to the stent. For example, as shown in FIG. 9, a coil 24 may be wrapped around the stent 10. Coil 24 may be closed looped and may be connected across cover 16 and reservoir 14, which may be embedded directly in stent 10. Alternatively, reservoir 14 may be etched in a substrate adhered to the stent. Cover 16, sealing the material 18 in the reservoir 14, may be configured consistent with any of the configurations, for example, shown in FIGS. 4A-7B, or any other suitable configuration.

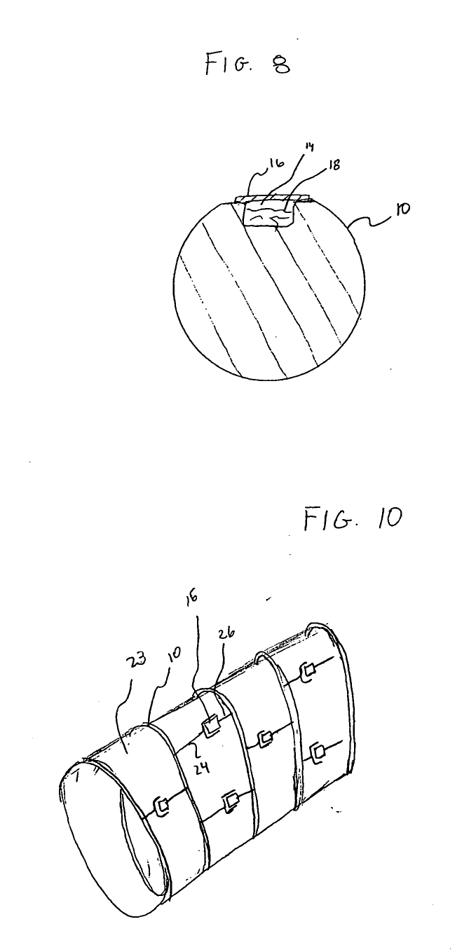

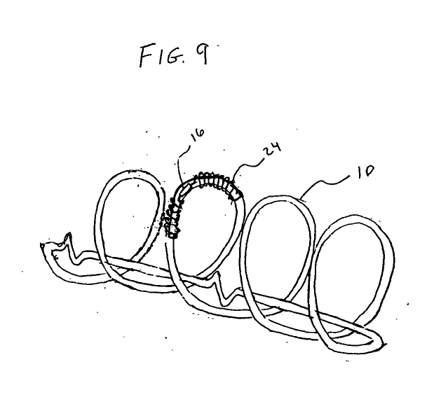

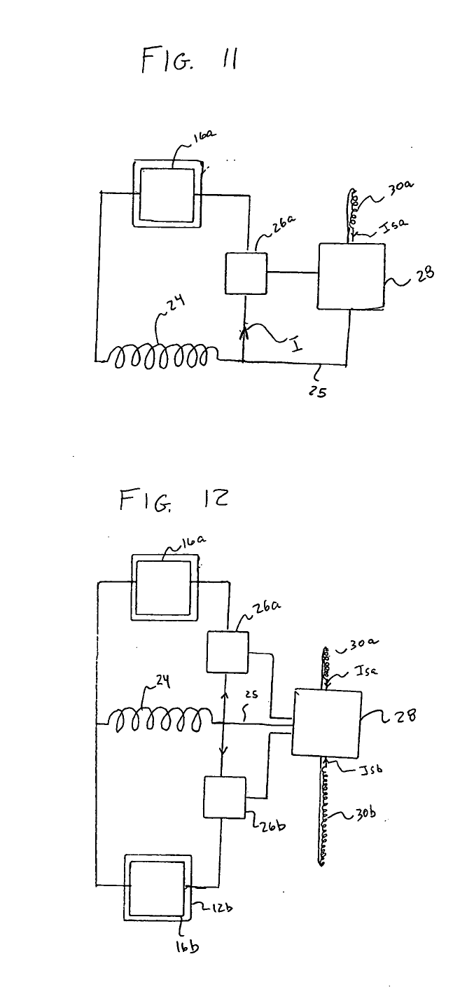

As shown in FIG. 10, reservoir 14 may also be embedded in a sleeve 23 disposed within or over stent 10, for example, a polymeric sleeve such as those used in a stent graft. Upon exposure of the stent 10 to a predetermined magnetic flux, current is drawn through anodes 24 and cathodes 26 from different portions of the conducting stent 10 across the cover 16. Reservoir 14 may also be embedded in a substrate or film, for example, made from a polymer, applied to the stent 10.

In an exemplary embodiment, the release of material 18 from reservoir 14 may be further controlled by one or more switches, for example, made from a semi-conductor material. The switches may be used to eliminate the effects of random foreign radio wave sources. FIG. 11 shows a switch 26a connected between a cover 16a and a primary coil 24. For clarity, the elements are shown in highly schematic form independent of the stent 10. Consistent with the embodiments detailed above, upon exposure of coil 24 to a first predetermined magnetic flux a current, I, is developed in coil 24 sufficient to open cover 16. Switch 26a is connected to a controller 28, such as a microprocessor, which may be connected to the substrate 12 or directly to the stent 10. Controller 28 may be powered inductively by the coil 24 via line 25 and may be configured to close switch 26a upon detection of a “key” second predetermined magnetic flux, for example, having a predetermined frequency and/or amplitude, which may be different than the first predetermined magnetic flux used to open cover 16. Upon exposure of secondary control coil 30a to the second predetermined magnetic flux, a current, Isa, develops in secondary control coil 30a, which serves as an input to the controller 28. Upon detection of input Isa, the controller 28 closes switch 26a allowing the cover 16 to be opened upon exposure of primary coil 24 to the first predetermined magnetic flux.

The use of switches may be expanded to control additional reservoirs. As can be seen in an exemplary embodiment shown in FIG. 12, two switches 26a and 26b may be used to control two covers 16a and 16b. Similar to the embodiment of FIG. 11, switch 26b may be used to prevent cover 16b from being opened until a “key” magnetic flux is detected by the controller 28. The “key” magnetic flux may be configured to generate a predetermined level of current, Isb, in secondary control coil 30b, which acts as a triggering input to controller 28. The opening of cover 16b is controlled in a similar manner to that of cover 16a, except that secondary control coil 30b may be configured to generate a triggering current upon exposure to a different “key” magnetic flux than that used to generate a triggering current in secondary control coil 30a. Consistently, secondary control coils 30a and 30b may have, for example, a different number of loops, a different loop size, etc. The opening of covers 16a, 16b may also be controlled by the same key magnetic flux, in which case, only one of the secondary control coils 30a and 30b may be used. Control coils 30a and 30 may also include a capacitor (not shown) connected across their ends, which may be used to tune each resonant circuit.

Switches 26a, 26b may be used, for example, to stagger the release of the material 18 from the reservoirs 14a, 14b (not shown). A staggered release may also be accomplished by using different primary coils for generating the current used to open the covers 16a, 16b. In such a case, different magnetic fluxes may be required to open each of the covers. Further, a staggered release may be accomplished by varying the material makeup of the covers such that they each require a different level of current, for example, to disintegrate or tear. In the case of the embodiment incorporating the heater 20, as shown in FIGS. 4A and 4B, the heat output of the heater 20 may vary from reservoir to reservoir. In the case of the embodiment incorporating a piezoelectric substrate 12 or cover 16, as shown in FIGS. 5A and 5B, the materials for the cover 16 and/or the substrate 12 may vary from reservoir to reservoir such that a first level of current is sufficient, for example, to open one cover but not the other. In the case of the embodiment of FIGS. 6A and 6B, different motors 24 may be used to pivot each cover 16 to an open position. The motors 24 may be actuatable by different levels of current.

In an exemplary embodiment of the present invention, rather than or in addition to having coils 30a and 30b, the controller 28 may be configured to detect modulation, such as amplitude modulation, of the first predetermined magnetic flux. The controller 28 may further be configured to open or close switch 26a so as to open or close cover 1 6a upon detection of a first modulation of the first predetermined magnetic flux. Similarly, controller 28 may be configured to open or close switch 26b so as to open or close cover 1 6b upon detection of a second modulation of the first predetermined magnetic flux, which may be different than the first modulation.

The present invention is not limited to stents. Further, the reservoirs may be filled with materials other than therapeutic agents. The reservoirs may be embedded in or connected to any device so long as the device permits access of the reservoir to the primary coil such that current generated in the primary coil passes through the reservoir or parts adjacent the reservoir used to open and close the reservoir. Non-limiting examples of medical devices according to the present invention include catheters, guide wires, balloons, filters (e.g., vena cava filters), stents, stent grafts, vascular grafts, intraluminal paving systems, and implants. Such medical devices may be implanted or otherwise utilized in body lumina and organs such as the coronary vasculature, esophagus, trachea, colon, biliary tract, urinary tract, prostate, brain, lung, liver, heart, skeletal muscle, kidney, bladder, intestines, stomach, pancreas, ovary, cartilage, eye, bone, and the like.

As indicated above, the reservoirs 14 may include a material 18, such as a drug. Further, medical devices according the present invention may also be coated with a drug. So as not to interfere with the opening of the covers, the coating may be performed in such a was so as to coat the entire body of the medical device except for the areas occupied by the reservoirs and their respective covers, etc.

The drug optionally stored in the reservoirs may be any pharmaceutically acceptable therapeutic agents such as non-genetic therapeutic agents, biomolecules, small molecules, or cells. Exemplary non-genetic therapeutic agents include anti-thrombogenic agents such heparin, heparin derivatives, prostaglandin (including micellar prostaglandin E1), urokinase, and PPack (dextrophenylalanine proline arginine chloromethylketone); anti-proliferative agents such as enoxaprin, angiopeptin, sirolimus (rapamycin), tacrolimus, everolimus, monoclonal antibodies capable of blocking smooth muscle cell proliferation, hirudin, and acetylsalicylic acid; anti-inflammatory agents such as dexamethasone, rosiglitazone, prednisolone, corticosterone, budesonide, estrogen, estrodiol, sulfasalazine, acetylsalicylic acid, mycophenolic acid, and mesalamine; anti-neoplastic/anti-proliferative/anti-mitotic agents such as paclitaxel, epothilone, cladribine, 5-fluorouracil, methotrexate, doxorubicin, daunorubicin, cyclosporine, cisplatin, vinblastine, vincristine, epothilones, endostatin, trapidil, halofuginone, and angiostatin; anti-cancer agents such as antisense inhibitors of c-myc oncogene; anti-microbial agents such as triclosan, cephalosporins, aminoglycosides, nitrofurantoin, silver ions, compounds, or salts; biofilm synthesis inhibitors such as non-steroidal anti-inflammatory agents and chelating agents such as ethylenediaminetetraacetic acid, O,O′-bis (2-aminoethyl)ethyleneglycol-N,N,N′,N′-tetraacetic acid and mixtures thereof; antibiotics such as gentamycin, rifampin, minocyclin, and ciprofolxacin; antibodies including chimeric antibodies and antibody fragments; anesthetic agents such as lidocaine, bupivacaine, and ropivacaine; nitric oxide; nitric oxide (NO) donors such as linsidomine, molsidomine, L-arginine, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, warfarin sodium, Dicumarol, aspirin, prostaglandin inhibitors, platelet aggregation inhibitors such as cilostazol and tick antiplatelet factors; vascular cell growth promotors such as growth factors, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogenous vascoactive mechanisms; inhibitors of heat shock proteins such as geldanamycin; angiotensin converting enzyme (ACE) inhibitors; beta-blockers; bAR kinase (bARKct) inhibitors; phospholamban inhibitors; protein-bound particle drugs such as ABRAXANE™; and any combinations and prodrugs of the above.

Exemplary biomolecules include peptides, polypeptides and proteins; oligonucleotides; nucleic acids such as double or single stranded DNA (including naked and cDNA), RNA, antisense nucleic acids such as antisense DNA and RNA, small interfering RNA (siRNA), and ribozymes; genes; carbohydrates; angiogenic factors including growth factors; cell cycle inhibitors; and anti-restenosis agents. Nucleic acids may be incorporated into delivery systems such as, for example, vectors (including viral vectors), plasmids or liposomes.

Non-limiting examples of proteins include serca-2 protein, monocyte chemoattractant proteins (“MCP-1) and bone morphogenic proteins (“BMP's”), such as, for example, BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15. Preferred BMPS are any of BMP-2, BMP-3, BMP-4, BMP-5, BMP-6, and BMP-7. These BMPs can be provided as homdimers, heterodimers, or combinations thereof, alone or together with other molecules. Alternatively, or in addition, molecules capable of inducing an upstream or downstream effect of a BMP can be provided. Such molecules include any of the “hedghog” proteins, or the DNA's encoding them. Non-limiting examples of genes include survival genes that protect against cell death, such as anti-apoptotic Bcl-2 family factors and Akt kinase; serca 2 gene; and combinations thereof. Non-limiting examples of angiogenic factors include acidic and basic fibroblast growth factors, vascular endothelial growth factor, epidermal growth factor, transforming growth factor α and β, platelet-derived endothelial growth factor, platelet-derived growth factor, tumor necrosis factor a, hepatocyte growth factor, and insulin like growth factor. A non-limiting example of a cell cycle inhibitor is a cathespin D (CD) inhibitor. Non-limiting examples of anti-restenosis agents include p15, p16, p18, p19, p21, p27, p53, p57, Rb, nFkB and E2F decoys, thymidine kinase (“TK”) and combinations thereof and other agents useful for interfering with cell proliferation.

Exemplary small molecules include hormones, nucleotides, amino acids, sugars, and lipids and compounds have a molecular weight of less than 100 kD.

Exemplary cells include stem cells, progenitor cells, endothelial cells, adult cardiomyocytes, and smooth muscle cells. Cells can be of human origin (autologous or allogenic) or from an animal source (xenogenic), or genetically engineered. Non-limiting examples of cells include side population (SP) cells, lineage negative (Lin⁻) cells including Lin⁻CD34⁻, Lin⁻CD34⁺, Lin⁻cKit⁺, mesenchymal stem cells including mesenchymal stem cells with 5-aza, cord blood cells, cardiac or other tissue derived stem cells, whole bone marrow, bone marrow mononuclear cells, endothelial progenitor cells, skeletal myoblasts or satellite cells, muscle derived cells, go cells, endothelial cells, adult cardiomyocytes, fibroblasts, smooth muscle cells, adult cardiac fibroblasts +5-aza, genetically modified cells, tissue engineered grafts, MyoD scar fibroblasts, pacing cells, embryonic stem cell clones, embryonic stem cells, fetal or neonatal cells, immunologically masked cells, and teratoma derived cells.

Any of the therapeutic agents may be combined to the extent such combination is biologically compatible.

The coating material of the medical device may include polymers, which may be biodegradable or non-biodegradable. Non-limiting examples of suitable non-biodegradable polymers include polystrene; polyisobutylene copolymers and styrene-isobutylene block copolymers such as styrene-isobutylene-styrene tri-block copolymers (SIBS); polyvinylpyrrolidone including cross-linked polyvinylpyrrolidone; polyvinyl alcohols, copolymers of vinyl monomers such as EVA; polyvinyl ethers; polyvinyl aromatics; polyethylene oxides; polyesters including polyethylene terephthalate; polyamides; polyacrylamides; polyethers including polyether sulfone; polyalkylenes including polypropylene, polyethylene and high molecular weight polyethylene; polyurethanes; polycarbonates, silicones; siloxane polymers; cellulosic polymers such as cellulose acetate; polymer dispersions such as polyurethane dispersions (BAYHDROL®); squalene emulsions; and mixtures and copolymers of any of the foregoing.

Non-limiting examples of suitable biodegradable polymers include polycarboxylic acid, polyanhydrides including maleic anhydride polymers; polyorthoesters; poly-amino acids; polyethylene oxide; polyphosphazenes; polylactic acid, polyglycolic acid and copolymers and mixtures thereof such as poly(L-lactic acid) (PLLA), poly(D,L,-lactide), poly(lactic acid-co-glycolic acid), 50/50 (DL-lactide-co-glycolide); polydioxanone; polypropylene fumarate; polydepsipeptides; polycaprolactone and co-polymers and mixtures thereof such as poly(D,L-lactide-co-caprolactone) and polycaprolactone co-butylacrylate; polyhydroxybutyrate valerate and blends; polycarbonates such as tyrosine-derived polycarbonates and arylates, polyiminocarbonates, and polydimethyltrimethylcarbonates; cyanoacrylate; calcium phosphates; polyglycosaminoglycans; macromolecules such as polysaccharides (including hyaluronic acid; cellulose, and hydroxypropylmethyl cellulose; gelatin; starches; dextrans; alginates and derivatives thereof), proteins and polypeptides; and mixtures and copolymers of any of the foregoing. The biodegradable polymer may also be a surface erodable polymer such as polyhydroxybutyrate and its copolymers, polycaprolactone, polyanhydrides (both crystalline and amorphous), maleic anhydride copolymers, and zinc-calcium phosphate.

Any of the above mentioned therapeutic agents may be used to fill the reservoirs, incorporated into a polymeric coating on the medical device or applied onto a polymeric coating on the medical device.

The material in the reservoirs of the present invention may be formed by any method known to one in the art. For example, an initial polymer/solvent mixture can be formed and then the therapeutic agent added to the polymer/solvent mixture. Alternatively, the polymer, solvent, and therapeutic agent can be added simultaneously to form the mixture. The polymer/solvent/therapeutic agent mixture may be a dispersion, suspension or a solution. The therapeutic agent may also be mixed with the polymer in the absence of a solvent. The therapeutic agent may be dissolved in the polymer/solvent mixture or in the polymer to be in a true solution with the mixture or polymer, dispersed into fine or micronized particles in the mixture or polymer, suspended in the mixture or polymer based on its solubility profile, or combined with micelle-forming compounds such as surfactants or adsorbed onto small carrier particles to create a suspension in the mixture or polymer. The material may comprise multiple polymers and/or multiple therapeutic agents.

Solvents may also be utilized in any order. For example, an initial polymer/solvent mixture can be formed and then the drug added to the polymer/solvent mixture. Alternatively, the polymer, solvent, and drug can be added simultaneously to form a mixture. Furthermore, multiple types of drug, polymers, and/or solvents may be utilized.

The medical device may also contain a radio-opacifying agent within its structure to facilitate viewing the medical device during insertion and at any point while the device is implanted. Non-limiting examples of radio-opacifying agents are bismuth subcarbonate, bismuth oxychloride, bismuth trioxide, barium sulfate, tungsten, and mixtures thereof.

The foregoing description and example have been set forth merely to illustrate the invention and are not intended as being limiting. Each of the disclosed aspects and embodiments of the present invention may be considered individually or in combination with other aspects, embodiments, and variations of the invention. None of the steps of the methods of the present invention are confined to any particular order of performance. Modifications of the disclosed embodiments incorporating the spirit and substance of the invention are within the scope of the present invention.

We claim:

1. A medical device comprising:
at least one primary coil;
at least one reservoir adapted to contain a material, said at least one reservoir connected to or embedded in the at least one primary coil;
a cover configured to seal the at least one reservoir in a first state of the cover and to allow the material to exit the at least one reservoir in a second state of the cover; and wherein said cover is configured such that it transitions between the first and second states when said coil is exposed to a magnetic flux.

2. The medical device of claim 1, wherein the cover in the second state is at least partially disintegrated.

3. The medical device of claim 1, wherein the cover in the second state completely covers the reservoir but is configured to allow the material to pass directly through it.

4. The medical device of claim 1, where the cover in the second state is shifted away from or towards the reservoir so as to unseal the reservoir.

5. The medical device of claim 1, wherein the cover in the second state has at least one smaller dimension than in the first state.

6. The medical device of claim 1, wherein the primary coil is connected such that a current induced in the primary coil by the magnetic flux passes through the cover.

7. The medical device of claim 1, wherein the medical device further comprises a substrate in which the at least one reservoir is formed.

8. The medical device of claim 1, wherein the medical device comprises a stent.

9. The medical device of claim 8, wherein the stent has a coil configuration and at least a portion of the coil configuration of the stent itself forms the primary coil.

10. The medical device of claim 8, wherein the primary coil is attached to a surface of the stent.

11. The medical device of claim 1, further comprising a second coil, wherein the at least one primary coil and the second coil are configured such that they each conduct a different level of current upon exposure to the same magnetic flux.

12. The medical device of claim 1, wherein said cover is a first cover and wherein said medical device further comprises a second reservoir covered by a second cover configured to seal the second reservoir in a first state and to allow material to exit the second reservoir in a second state, said first cover configured to transition between its first and second states when a first current passes through it, said second cover configured to transition between its first and second states when a second current different from the first current passes through it.

13. The medical device of claim 1, wherein the magnetic flux has a radio frequency.

14. The medical device of claim 1, further comprising at least one switch configured to control electrical communication between the cover and the primary coil.

15. The medical device of claim 14, further comprising a controller configured to control the at least one switch.

16. The medical device of claim 1, wherein the material is a therapeutic agent.

17. The medical device of claim 12, wherein the material is a first therapeutic agent and the second reservoir contains a second therapeutic agent.

18. The medical device of claim 1, wherein the at least one reservoir is formed in a polymer sleeve.

19. The medical device of claim 1, wherein the magnetic flux is time varying.
